(19) United States
(12) Patent Application Publication
(10) Pub. No.: US 2021/0139503 A1
(43) Pub. Date: May 13, 2021

(54) PROCESSES FOR PREPARING AN S1P-RECEPTOR MODULATOR

(71) Applicant: ESCAPE Bio, Inc., South San Francisco, CA (US)

(72) Inventors: Stéphane De Lombaert, Brisbane, CA (US); Jonathon Daryll Schwarz Holt, Durham, NC (US); Ana Rosario Mollo Sarno, San Mateo, CA (US); Jacob Bradley Schwarz, San Ramon, CA (US); Michael A. Christie, Phoenixville, PA (US); Edward L. Ciolkowski, Webster, NY (US); Zenghong Zhang, North Brunswick, NJ (US)

(21) Appl. No.: 17/084,715

(22) Filed: Oct. 30, 2020

Related U.S. Application Data
(60) Provisional application No. 62/928,422, filed on Oct. 31, 2019.

(51) Int. Cl. C07D 498/04

(52) U.S. Cl. CPC C07D 498/04

(57) ABSTRACT

This application relates to processes for preparing an S1P-receptor modulator, which is useful in the treatment of diseases or disorders associated with activity of S1P, including CNS disorders.

### FIELD OF THE INVENTION

This application relates to processes for preparing an S1P-receptor modulator, which is useful in the treatment of diseases or disorders associated with activity of S1P, including CNS disorders.

### BACKGROUND OF THE INVENTION

Sphingosine-1-phosphate (S1P) is a bioactive sphingolipid that mediates a wide variety of cellular responses, such as proliferation, cytoskeletal organization and migration, adherence- and tight junction assembly, and morphogenesis. S1P can bind with members of the endothelial cell differentiation gene family (EDG receptors) of plasma membrane-localized G protein-coupled receptors. To date, five members of this family have been identified as S1P receptors in different cell types, S1P1 (EDG-1), S1P2 (EDG-5), S1P3 (EDG-3), S1P4 (EDG-6) and S1P5 (EDG-8). S1P can produce cytoskeletal re-arrangements in many cell types to regulate immune cell trafficking, vascular homeostasis and cell communication in the central nervous system (CNS) and in peripheral organ systems.

It is known that S1P is secreted by vascular endothelium and is present in blood at concentrations of 200-900 nanomolar and is bound by albumin and other plasma proteins. This provides both a stable reservoir in extracellular fluids and efficient delivery to high-affinity cell-surface receptors. S1P binds with low nanomolar affinity to the five receptors S1P1-5. In addition, platelets also contain S1P and may be locally released to cause e.g. vasoconstriction. The receptor subtypes S1P1, S1P2 and S1P3 are widely expressed and represent dominant receptors in the cardiovascular system. Further, S1P1 is also a receptor on lymphocytes. S1P4 receptors are almost exclusively in the haematopoietic and lymphoid system. S1P5 is primarily (though not exclusively) expressed in central nervous system. The expression of S1P5 appears to be restricted to oligodendrocytes in mice, the myelinating cells of the brain, while in rat and man expression at the level of astrocytes and endothelial cells was found but not on oligodendrocytes.

S1P receptor modulators are compounds which signal as (ant)agonists at one or more S1P receptors. The present invention relates to modulators of the S1P5 receptor, in particular agonists, and preferably to agonists with selectivity over S1P1 and/or S1P3 receptors, in view of unwanted cardiovascular and/or immunomodulatory effects. It has now been found that S1P5 agonists can be used in the treatment of cognitive disorders, in particular age-related cognitive decline.

Research is ongoing to develop therapeutics that can be used to treat age related cognitive decline and dementia. For example, the compound (1s,3s)-3-(2-(4-((4-chlorobenzyl)oxy)phenyl)-6,7-dihydrooxazolo[4,5-c]pyridin-5(4H)-yl)cyclobutane-1-carboxylic acid (Compound 1) and other small molecule modulators of the S1P receptors are reported in, e.g., U.S. Pat. No. 8,796,262. There is a need for improved methods of preparing S1P-receptor modulators like Compound 1 in order, for example, to increase purity, improve reproducibility, improve efficiency, reduce costs, and allow for scale up. The present disclosure helps fulfill these and other needs, as evident in reference to the following disclosure.

### SUMMARY OF THE INVENTION

Provided herein are processes for preparing (1s,3s)-3-(2-(4-((4-chlorobenzyl)oxy)phenyl)-6,7-dihydrooxazolo[4,5-c]pyridin-5(4H)-yl)cyclobutane-1-carboxylic acid (“Compound 1”) and salts thereof. Provided herein are also intermediates useful for the preparation of Compound 1 and salts thereof.

Provided herein are also pharmaceutical compositions, which include Compound 1 and pharmaceutically acceptable salts thereof, and one or more pharmaceutically acceptable carriers or excipients.

The present disclosure also provides methods of modulating S1P receptor (e.g., S1P5) activity, comprising contacting Compound 1 or a pharmaceutically acceptable salt thereof with an S1P receptor. The present invention further provides a method for treating a CNS disorder in a patient, comprising: administering to the patient a therapeutically effective amount of Compound 1, or a pharmaceutically acceptable salts thereof.

The present disclosure also provides therapeutic methods of using Compound 1 and pharmaceutically acceptable salts thereof. The present disclosure also provides uses of Compound 1 and pharmaceutically acceptable salts thereof in the manufacture of a medicament for use in therapy. The present disclosure also provides Compound 1 and pharmaceutically acceptable salts thereof for use in therapy.

### BRIEF DESCRIPTION OF THE DRAWINGS

FIG. 1 shows a nuclear magnetic resonance (NMR) spectrum of Compound 1.

FIG. 2 shows an NMR spectrum of Compound 5.

### DETAILED DESCRIPTION

This disclosure provides processes and intermediates for preparing a S1P-receptor modulator. The present disclosure is directed to, inter alia, processes for preparing (1s,3s)-3-(2-(4-((4-chlorobenzyl)oxy)phenyl)-6,7-dihydrooxazolo[4,5-c]pyridin-5(4H)-yl)cyclobutane-1-carboxylic acid (Compound 1) or a salt thereof. The structure of Compound 1 is shown below.

[chemical structure]

Compound 1 is described in U.S. Pat. No. 8,796,262, the entirety of which is incorporated herein by reference. The processes for preparing Compound 1 or a salt thereof provided herein have certain advantages over the processes currently disclosed in the art. For example, the processes described herein demonstrate good scalability and yields. In particular, the reactions are easily handled because each step of the process provides a filterable solid. In addition, the processes described herein demonstrate good selectivity for the cis-isomer of Compound 1.

Provided herein is a process for preparing Compound 1 having the formula:

[chemical structure]

or a salt thereof, comprising reacting Compound 2, having the formula:

[chemical structure]

with Compound 3, having the formula:

[chemical structure]

in the presence of RA1, wherein RA1 is a reducing agent, to provide Compound 1, or a salt thereof.

RA1 can be a hydride reducing agent. In some embodiments, RA1 is sodium cyanoborohydride.

The reacting of Compound 2 with Compound 3 can be performed in the presence of S1, wherein S1 is a protic solvent. In some embodiments, S1 is methanol.

In some embodiments, the reacting of Compound 2 with Compound 3 comprises using about 1 to about 5 molar equivalents of Compound 3 relative to Compound 2. In some embodiments, the reacting of Compound 2 with Compound 3 comprises using about 1 to about 2 molar equivalents of Compound 3 relative to Compound 2. In some embodiments, the reacting of Compound 2 with Compound 3 comprises using about 1.25 molar equivalents of Compound 3 relative to Compound 2. In some embodiments, the reacting of Compound 2 with Compound 3 comprises using about 1 to about 5 molar equivalents of RA1 relative to Compound 2. In some embodiments, the reacting of Compound 2 with Compound 3 comprises using about 1 to about 3 molar equivalents of RA1 relative to Compound 2. In some embodiments, the reacting of Compound 2 with Compound 3 comprises using about 2 to about 3 molar equivalents of RA1 relative to Compound 2. In some embodiments, the reacting of Compound 2 with Compound 3 comprises using about 2.2 molar equivalents of RA1 relative to Compound 2.

The reacting of Compound 2 with Compound 3 can be performed at a temperature of about 20° C. to about 35° C. In some embodiments, the reacting of Compound 2 with Compound 3 is performed at a temperature of about 20° C. to about 35° C. In some embodiments, the reacting of Compound 2 with Compound 3 is performed at room temperature.

In some embodiments, the process further comprises precipitating Compound 1 from a solution comprising Compound 1 and Sla, wherein Sla is an aprotic solvent. In some embodiments, Sla is DMSO. In some embodiments, the precipitating of Compound 1 from a solution comprising Compound 1 and Sla comprises 1) heating the solution to a first temperature, and 2) cooling the solution to a second temperature. In some embodiments, the first temperature is between about 50° C. and about 80° C. In some embodiments, the first temperature is between about 65° C. and about 75° C. In some embodiments, the first temperature is about 70° C. In some embodiments, the second temperature is between about 15° C. and about 25° C. In some embodiments, the second temperature is about 20° C. In some embodiments, Compound 1 is precipitated as a crystalline solid.

In some embodiments, the process further comprises precipitating Compound 1 from a solution comprising Compound 1 and S1b, wherein S1b is a mixture of a protic solvent and A1. In some embodiments, S1b is a mixture of water and A1. In some embodiments, A1 is an organic acid. In some embodiments, A1 is acetic acid. In some embodiments, the precipitating of Compound 1 from a solution comprising Compound 1 and S1b comprises 1) heating the solution to a first temperature, and 2) cooling the solution to a second temperature. In some embodiments, the first temperature is between about 50° C. and about 80° C. In some embodiments, the first temperature is between about 65° C. and about 75° C. In some embodiments, the first temperature is about 70° C. In some embodiments, the second temperature is between about 15° C. and about 25° C. In some embodiments, the second temperature is about 20° C. In some embodiments, Compound 1 is precipitated as a crystalline solid.

Compound 2 can be prepared by a process comprising reacting Compound 4, having the formula:

[chemical structure]

with A2, wherein A2 is an acid.

In some embodiments, A2 is an organic acid. In some embodiments, A2 is trifluoroacetic acid.

The reacting of Compound 4 with A2 can be performed in the presence of S2, wherein S2 is a halogenated solvent. In some embodiments, S2 is methylene chloride.

In some embodiments, the reacting of Compound 4 with A2 comprises using about 1 to about 50 molar equivalents of A2 relative to Compound 4. In some embodiments, the reacting of Compound 4 with A2 comprises using about 1 to about 20 molar equivalents of A2 relative to Compound 4. In some embodiments, the reacting of Compound 4 with A2 comprises using about 5 to about 15 molar equivalents of A2 relative to Compound 4. In some embodiments, the reacting of Compound 4 with A2 comprises using about 10 molar equivalents of A2 relative to Compound 4.

The reacting of Compound 4 with Compound A2 can be performed at a temperature of about 20° C. to about 30° C. In some embodiments, the reacting of Compound 4 with Compound A2 is performed at room temperature.

Compound 4 can be prepared by a process comprising reacting Compound 5, having the formula:

[chemical structure]

with a compound of Formula II:

[chemical structure]

wherein X is Br, Cl, or I;

in the presence of B1, wherein B1 is a base.

In some embodiments, X is Br or Cl. In some embodiments, X is Br. In some embodiments, X is Cl. In some embodiments, X is I. In some embodiments, the compound of Formula II is p-chlorobenzyl bromide. In some embodiments, the compound of Formula II is p-chlorobenzyl chloride.

In some embodiments, B1 is a carbonate base. In some embodiments, B1 is potassium carbonate.

The reacting of Compound 5 with the compound of Formula II in the presence of B1 can be performed in the presence of S3, wherein S3 is a polar aprotic solvent. In some embodiments, S3 is acetonitrile.

In some embodiments, the reacting of Compound 5 with the compound of Formula II in the presence of B1 comprises using about 1 to about 5 molar equivalents of p the compound of Formula II relative to Compound 5. In some embodiments, the reacting of Compound 5 with the compound of Formula II in the presence of B1 comprises using about 1 to about 2 molar equivalents of the compound of Formula II relative to Compound 5. In some embodiments, the reacting of Compound 5 with the compound of Formula II in the presence of B1 comprises using about 1.1 molar equivalent of the compound of Formula II relative to Compound 5. In some embodiments, the reacting of Compound 5 with the compound of Formula II in the presence of B1 comprises using about 1 to about 5 molar equivalents of B1 relative to Compound 5. In some embodiments, the reacting of Compound 5 with the compound of Formula II in the presence of B1 comprises using about 1 to about 2 molar equivalents of B1 relative to Compound 5. In some embodiments, the reacting of Compound 5 with the compound of Formula II in the presence of B1 comprises using about 1.1 molar equivalent of B1 relative to Compound 5.

The reacting of Compound 5 with the compound of Formula II in the presence of B1 can be performed at a temperature between about 30° C. and about 60° C. In some embodiments, the reacting of Compound 5 with the compound of Formula II in the presence of B1 is performed at a temperature between about 45° C. and about 55° C. In some embodiments, the reacting of Compound 5 with the compound of Formula II in the presence of B1 is performed at a temperature of about 50° C.

Compound 5 can be prepared by a process comprising reacting Compound 6 having the formula:

[chemical structure]

with B4, wherein B4 is a base.

In some embodiments, B4 is a metal hydroxide base. In some embodiments, B4 is sodium hydroxide.

The reacting of Compound 6 with B4 can be performed in the presence of S4, wherein S4 is a polar solvent. In some embodiments, S4 is water.

The preparation of Compound 5 can further comprise precipitating Compound 5 from a mixture comprising S4a, wherein S4a is a polar protic solvent. In some embodiments, S4a is an alcohol. In some embodiments, S4a is ethanol.

Compound 6 can be prepared by a process comprising reacting Compound 7 having the formula:

[chemical structure]

with P1, wherein P1 is a phosphorous reagent.

In some embodiments, P1 is triphenylphosphine or phosphorous tribromide. In some embodiments, P1 is triphenylphosphine. In some embodiments, P1 is phosphorous tribromide.

The reacting of Compound 7 with P1 can be performed in the presence of S5, wherein S5 is a polar aprotic solvent. In some embodiments, S5 is acetonitrile. In some embodiments, S5 is THF.

In some embodiments, the reacting of Compound 7 with P1 is further performed in the presence of an additive. The additive can be, e.g., pyridine, hexachloroethane, or a mixture thereof.

Compound 7 can be prepared by a process comprising reacting Compound 8 having the formula:

[chemical structure]

wherein R is C₁₋₂ alkyl;

with A3, wherein A3 is an acid.

In some embodiments, each R is methyl. In some embodiments, each R is ethyl.

In some embodiments, A3 is a mineral acid. In some embodiments, A3 is HCl.

The reacting of Compound 8 with A3 can be performed in the presence of S6, wherein S6 is a polar aprotic solvent. In some embodiments, S6 is ethyl acetate.

Compound 8 can be prepared by a process comprising reacting Compound 9 having the formula:

[chemical structure]

with Compound 12 having the formula:

[chemical structure]

wherein R is C₁₋₂ alkyl, in the presence of B3, wherein B3 is a base.

In some embodiments, each R is methyl. In some embodiments, each R is ethyl.

In some embodiments, B3 is an organic base. In some embodiments, B3 is triethylamine.

The reacting of Compound 9 with Compound 12 can be performed in the presence of S7, wherein S7 is a polar aprotic solvent. In some embodiments, S7 is ethyl acetate.

Compound 9 can be prepared by a process comprising reacting Compound 10 having the formula:

[chemical structure]

with Cl1, wherein Cl1 is a chlorinating reagent.

In some embodiments, Cl1 is oxalyl chloride.

The reacting of Compound 10 with Cl1 can be performed in the presence of S8, wherein S8 is a polar aprotic solvent. In some embodiments, S8 is dichloromethane.

The reacting of Compound 10 with Cl1 can be performed at a temperature between about 20° C. and about 30° C. The reacting of Compound 10 with Cl1 can be performed at about room temperature.

Compound 10 can be prepared by a process comprising reacting compound 11, having the formula:

[chemical structure]

with acetic anhydride in the presence of B2, wherein B2 is a base.

In some embodiments, B2 is an organic base. In some embodiments, B2 is triethylamine.

The reacting of Compound 11 with acetic anhydride can be performed at a temperature between about 80° C. and about 120° C. The reacting of Compound 11 with acetic anhydride can be performed at a temperature between about 90° C. and about 100° C. The reacting of Compound 11 with acetic anhydride can be performed at a temperature of about 100° C.

Compound 12 can be prepared by a process comprising reacting Compound 13 having the formula:

[chemical structure]

with sodium C₁₋₂ alkoxide.

In some embodiments, the sodium C₁₋₂ alkoxide is sodium methoxide. In some embodiments, the sodium C₁₋₂ alkoxide is sodium ethoxide.

The reacting of Compound 13 with sodium C₁₋₂ alkoxide can be performed in the presence of S10, wherein S10 is a polar protic solvent. In some embodiments, S10 is an alcohol.

In some embodiments, S10 is methanol or ethanol. In some embodiments, S10 is methanol. In some embodiments, S10 is ethanol. In some embodiments, when the sodium C₁₋₂ alkoxide is sodium methoxide, then S10 is methanol. In some embodiments, when the sodium C₁₋₂ alkoxide is sodium ethoxide, then S10 is ethanol.

The reacting of Compound 13 with sodium C₁₋₂ alkoxide can be performed at a temperature between about 0° C. and about 20° C. The reacting of Compound 13 with sodium C₁₋₂ alkoxide can be performed at a temperature between about 5° C. and about 15° C. The reacting of Compound 13 with sodium C₁₋₂ alkoxide can be performed at a temperature of about 10° C.

Compound 13 can be prepared by a process comprising reacting Compound 14 having the formula:

[chemical structure]

with tosyl chloride in the presence of B6, wherein B6 is a base.

In some embodiments, B6 is an organic base. In some embodiments, B6 is triethylamine.

The reacting of Compound 14 with tosyl chloride can be performed in the presence of S11, wherein S1l is a solvent. In some embodiments, S1l is a polar aprotic solvent. In some embodiments, S11 is dichloromethane.

Compound 14 can be prepared by a process comprising reacting Compound 15 having the formula:

[chemical structure]

with hydroxylamine, or a salt thereof, in the presence of B5, wherein B5 is a base.

In some embodiments, B5 is a metal hydroxide base. In some embodiments, B5 is sodium hydroxide.

In some embodiments, the hydroxylamine, or a salt thereof, is the HCl salt of hydroxylamine.

The reacting of Compound 15 with B5 can be performed in the presence of S12, wherein S12 is a solvent. In some embodiments, S12 is a mixture of polar protic solvents. In some embodiments, S12 is a mixture of water and an alcohol. In some embodiments, S12 is a mixture of water and methanol.

The reacting of Compound 15 with B5 can be performed at a temperature between about 20° C. and about 30° C. The reacting of Compound 15 with B5 can be performed at room temperature.

Provided herein is a process for preparing Compound 1 having the formula:

[chemical structure]

or a salt thereof, comprising:
a) reacting Compound 5, having the formula:

[chemical structure]

with a compound of Formula II:

[chemical structure]

wherein X is Br, Cl, or I; in the presence of B1, wherein B1 is a base, to provide Compound 4 having the formula:

[chemical structure]

b) reacting Compound 4 with A2, wherein A2 is an acid to provide Compound 2, having the formula:

[chemical structure]

and
c) reacting Compound 2 with Compound 3, having the formula:

[chemical structure]

in the presence of RA1, wherein RA1 is a reducing agent, to provide Compound 1, or a salt thereof.

Provided herein is a process for preparing Compound 1 having the formula:

[chemical structure]

or a salt thereof, comprising:
a) reacting Compound 4, having the formula:

[chemical structure]

with A2, wherein A2 is an acid to provide Compound 2, having the formula:

[chemical structure]

and
b) reacting Compound 2 with Compound 3, having the formula:

[chemical structure]

in the presence of RA1, wherein RA1 is a reducing agent, to provide Compound 1, or a salt thereof.

Provided herein is a process for preparing Compound 5 having the formula:

[chemical structure]

or a salt thereof, comprising:
a) reacting compound 11, having the formula:

[chemical structure]

with acetic anhydride in the presence of B2, wherein B2 is a base, to provide Compound 10 having the formula:

[chemical structure]

b) reacting Compound 10 with Cl1, wherein Cl1 is a chlorinating reagent, to provide Compound 9 having the formula:

[chemical structure]

c) reacting Compound 9 with Compound 12 having the formula:

[chemical structure]

wherein R is C₁₋₂ alkyl, in the presence of B3, wherein B3 is a base, to provide Compound 8 having the formula:

[chemical structure]

wherein R is C₁₋₂ alkyl;
d) reacting Compound 8 with A3, wherein A3 is an acid, to provide Compound 7 having the formula:

[chemical structure]

e) reacting Compound 7 with P1, wherein P1 is a phosphorous reagent, to provide Compound 6 having the formula:

[chemical structure]

f) reacting Compound 6 with B4, wherein B4 is a base, to provide Compound 5.

Provided herein is a process for preparing Compound 12 having the formula:

[chemical structure]

or a salt thereof, wherein R is C₁₋₂ alkyl, comprising:
a) reacting Compound 15 having the formula:

[chemical structure]

with hydroxylamine in the presence of B5, wherein B5 is a base, to provide Compound 14 having the formula:

[chemical structure]

b) reacting Compound 14 with tosyl chloride in the presence of B6, wherein B6 is a base, to provide Compound 13 having the formula:

[chemical structure]

c) reacting Compound 13 with sodium C₁₋₂ alkoxide (e.g., sodium methoxide or sodium ethoxide) to provide Compound 12.

Compound 1 can be isolated as one or more solid forms. The solid forms (e.g., crystalline forms) described herein can have certain advantages, for example, they may have desirable properties, such as ease of handling, ease of processing, storage stability, and ease of purification. Moreover, the crystalline forms can be useful for improving the performance characteristics of a pharmaceutical product such as intrinsic solubility, dissolution profile, shelf-life and bioavailability.

It is appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination.

As used herein, the term “reacting,” “contacting” or “treating” when describing a certain process is used as known in the art and generally refers to the bringing together of chemical reagents in such a manner so as to allow their interaction at the molecular level to achieve a chemical or physical transformation. In some embodiments, the reacting involves two reagents, wherein one or more equivalents of second reagent are used with respect to the first reagent. The reacting steps of the processes described herein can be conducted for a time and under conditions suitable for preparing the identified product.

As used herein, the terms “converting” with respect to changing an intermediate or starting reagent or material in a chemical reaction refers to subjecting the intermediate or starting reagent or material to the suitable reagents and conditions (e.g., temperature, time, solvent, etc.) to effect certain changes (e.g., breaking or formation of a chemical bond) to generate the desired product.

As used herein, and unless otherwise specified, the term “about”, when used in connection with a numeric value or range of values which is provided to describe a particular compound of reaction (e.g., a specific temperature or temperature range, such as describing a heating, cooling, melting, dehydration, or glass transition; a mass change, such as a mass change as a function of temperature or humidity; a solvent or water content, in terms of, for example, mass or a percentage; or a peak position, such as in analysis by, for example, ¹³C NMR, indicate that the value or range of values may deviate to an extent deemed reasonable to one of ordinary skill in the art while still describing the particular compound or reaction. Specifically, the term “about”, when used in this context, indicates that the numeric value or range of values may vary by 5%, 4%, 3%, 2%, 1%, 0.9%, 0.8%, 0.7%, 0.6%, 0.5%, 0.4%, 0.3%, 0.2% or 0.1% of the recited value or range of values while still describing the particular compound or reaction.

The term “organic acid” refers to an acid with an organic moiety. Examples of organic acid include but not limited to acetic acid, trifluoroacetic acid, formic acid, benzoic acid, toluenesulfonic acid, triflic acid, and the like.

The term “carbonate base” refers to a base containing a carbonate group. Examples of carbonate bases include but are not limited to sodium carbonate, potassium carbonate, and the like.

As used herein, the phrase “metal hydroxide base,” employed alone or in combination with other terms, refers to a base having formula MOH, wherein M refers to a metal such as an alkali metal (e.g. lithium, sodium, or potassium). Example alkali metal hydroxide bases include, but are not limited to lithium hydroxide, sodium hydroxide, and potassium hydroxide.

The term “organic base” refers to a base with an organic moiety. Examples of organic base include but not limited to triethylamine.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected. In some embodiments, reactions can be carried out in the absence of solvent, such as when at least one of the reagents is a liquid or gas.

Suitable solvents can include halogenated solvents such as carbon tetrachloride, bromodichloromethane, dibromochloromethane, bromoform, chloroform, bromochloromethane, dibromomethane, butyl chloride, dichloromethane (methylene chloride), tetrachloroethylene, trichloroethylene, 1,1,1-trichloroethane, 1,1,2-trichloroethane, 1,1-dichloroethane, 2-chloropropane, α,α,α-trifluorotoluene, 1,2-dichloroethane, 1,2-dibromoethane, hexafluorobenzene, 1,2,4-trichlorobenzene, 1,2-dichlorobenzene, chlorobenzene, fluorobenzene, mixtures thereof and the like.

Suitable ether solvents include: dimethoxymethane, tetrahydrofuran, cyclopentyl methyl ether, 1,3-dioxane, 1,4-dioxane, furan, tetrahydrofuran (THF), diethyl ether, ethylene glycol dimethyl ether, ethylene glycol diethyl ether, diethylene glycol dimethyl ether (diglyme), diethylene glycol diethyl ether, triethylene glycol dimethyl ether, anisole, methyl tert-butyl ether, mixtures thereof and the like.

Suitable polar protic solvents can include, by way of example and without limitation, water, methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, iso-butyl alcohol, tert-butyl alcohol, 2-ethoxyethanol, diethylene glycol, 1-, 2-, or 3-pentanol, neo-pentyl alcohol, tert-pentyl alcohol, diethylene glycol monomethyl ether, diethylene glycol monoethyl ether, cyclohexanol, benzyl alcohol, phenol, or glycerol. The polar protic solvent can be an alcohol such as methanol, ethanol, 1-propanol, 2-propanol, and the like.

Suitable aprotic solvents can include, by way of example and without limitation, 2-butanone, acetonitrile, dichloromethane, N,N-dimethylformamide (DMF), N,N-dimethylacetamide (DMA), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, dimethyl sulfoxide, propionitrile, ethyl formate, methyl acetate, hexachloroacetone, acetone, ethyl methyl ketone, ethyl acetate, sulfolane, N,N-dimethylpropionamide, tetramethylurea, nitromethane, nitrobenzene, hexamethylphosphoramide, and the like. Suitable polar aprotic solvents can include, by way of example, acetonitrile, dichloromethane, and ethyl acetate.

Suitable hydrocarbon solvents include benzene, cyclohexane, pentane, hexane, toluene, cycloheptane, methylcyclohexane, heptane, ethylbenzene, m-, o-, or p-xylene, octane, indane, nonane, or naphthalene.

The term “reducing agent” as used herein refers to a compound that donates an electron to another compound in a redox reaction. The reducing agent would be oxidized after it loses its electrons. Examples of reducing agents include, but not limited to, borohydride, triacetoxyborohydride, sodium borohydride, lithium aluminum hydride, hydrogen on palladium, and the like. The reducing agent can be a hydride reducing agent. Hydride reducing agents are reducing agents that contain one or more hydrogen centers having reducing properties. Example hydride reducing agents include, but are not limited to, lithium aluminum hydride, sodium borohydride, sodium triacetoxyborohydride, sodium cyanoborohydride, and the like.

The reactions of the processes described herein can be carried out in air or under an inert atmosphere. Typically, reactions containing reagents or products that are substantially reactive with air can be carried out using air-sensitive synthetic techniques that are well known to the skilled artisan.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry; or by chromatography such as high performance liquid chromatography (HPLC) or thin layer chromatography. The compounds obtained by the reactions can be purified by any suitable method known in the art. For example, chromatography (medium pressure) on a suitable adsorbent (e.g., silica gel, alumina and the like), HPLC, or preparative thin layer chromatography; distillation; sublimation, trituration, or recrystallization. The purity of the compounds, in general, are determined by physical methods such as measuring the melting point (in case of a solid), obtaining a NMR spectrum, or performing a HPLC separation. If the melting point decreases, if unwanted signals in the NMR spectrum are decreased, or if extraneous peaks in an HPLC trace are removed, the compound can be said to have been purified. In some embodiments, the compounds are substantially purified.

The reactions of the processes described herein can be carried out at appropriate temperatures which can be readily determined by the skilled artisan. Reaction temperatures will depend on, for example, the melting and boiling points of the reagents and solvent, if present; the thermodynamics of the reaction (e.g., vigorously exothermic reactions may need to be carried out at reduced temperatures); and the kinetics of the reaction (e.g., a high activation energy barrier may need elevated temperatures).

The expressions, “ambient temperature” and “room temperature,” as used herein, are understood in the art, and refer generally to a temperature, e.g., a reaction temperature, that is about the temperature of the room in which the reaction is carried out, for example, a temperature from about 20° C. to about 30° C.

As used herein, the phrase “solid form” refers to a compound provided herein in either an amorphous state or a crystalline state (“crystalline form” or “crystalline solid” or “crystalline solid form”), whereby a compound provided herein in a crystalline state may optionally include solvent or water within the crystalline lattice, for example, to form a solvated or hydrated crystalline form. In some embodiments, the compound provided herein is in a crystalline state as described herein.

Compounds provided herein (e.g., Compound 1) can also include all isotopes of atoms occurring in the intermediates or final compounds. Isotopes include those atoms having the same atomic number but different mass numbers. For example, isotopes of hydrogen include tritium and deuterium. One or more constituent atoms of the compounds provided herein can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7 or 8 deuterium atoms. Synthetic methods for including isotopes into organic compounds are known in the art.

In some embodiments, Compound 1 is substantially isolated. The term “substantially isolated” is meant that the compound is at least partially or substantially separated from the environment in which it was formed or detected. Partial separation can include, e.g., a composition enriched in the compound, salts, hydrates, solvates, or solid forms provided herein. Substantial separation can include compositions containing at least about 50%, at least about 60%, at least about 70%, at least about 80%, at least about 90%, at least about 95%, at least about 97%, or at least about 99% by weight of the compound, salts, hydrates, solvates, or solid forms thereof.

In some embodiments, concentrating a solution as described herein refers to a solution where its volume is reduced by letting the solvent evaporate, by heating the solution, by subjecting the solution to reduced pressure, or any combination thereof.

### Compounds

Provided herein is Compound 1 having the formula:

[chemical structure]

or a salt thereof.

Provided herein is Compound 2 having the formula:

[chemical structure]

or a salt thereof.

Provided herein is Compound 3 having the formula:

[chemical structure]

or a salt thereof.

Provided herein is a compound of Formula Ia:

[chemical structure]

or a salt thereof, wherein R¹ is H, C₁₋₆ alkyl, C₃₋₇ cycloalkyl, phenyl, 4-7 membered heterocycloalkyl, or 5-6 membered heteroaryl, wherein the C₁₋₆ alkyl is optionally substituted by 1-5 halo, or optionally substituted by a C₃₋₇ cycloalkyl, phenyl, 4-7 membered heterocycloalkyl, or 5-6 membered heteroaryl group. In some embodiments, R¹ is C₁₋₆ alkyl.

Provided herein is Compound 4 having the formula:

[chemical structure]

or a salt thereof.

Provided herein is a compound of Formula Ib:

[chemical structure]

or a salt thereof, wherein R¹ is H, C₁₋₆ alkyl, C₃₋₇ cycloalkyl, phenyl, 4-7 membered heterocycloalkyl, or 5-6 membered heteroaryl, wherein the C₁₋₆ alkyl is optionally substituted by 1-5 halo, or optionally substituted by a C₃₋₇ cycloalkyl, phenyl, 4-7 membered heterocycloalkyl, or 5-6 membered heteroaryl group. In some embodiments, R¹ is C₁₋₆ alkyl.

Provided herein is Compound 5 having the formula:

[chemical structure]

or a salt thereof.

Provided herein is Compound 6 having the formula:

[chemical structure]

or a salt thereof.

Provided herein is Compound 7 having the formula:

[chemical structure]

or a salt thereof.

Provided herein is Compound 8 having the formula:

[chemical structure]

or a salt thereof, wherein R is methyl or ethyl. In some embodiments, each R is methyl. In some embodiments, each R is ethyl.

Provided herein is Compound 9 having the formula:

[chemical structure]

or a salt thereof.

Provided herein is Compound 10 having the formula:

[chemical structure]

or a salt thereof.

Provided herein is Compound 11 having the formula:

[chemical structure]

or a salt thereof.

Provided herein is Compound 12 having the formula:

[chemical structure]

or a salt thereof, wherein R is methyl or ethyl. In some embodiments, R is methyl. In some embodiments, R is ethyl.

Provided herein is Compound 13 having the formula:

[chemical structure]

or a salt thereof.

Provided herein is Compound 14 having the formula:

[chemical structure]

or a salt thereof.

Provided herein is Compound 15 having the formula:

[chemical structure]

or a salt thereof.

As used herein, the term “BOC” refers to the N-protecting group tert-butyloxycarbonyl.

As used herein, the term “alkyl,” employed alone or in combination with other terms, refers to a saturated hydrocarbon group that may be straight-chain or branched. In some embodiments, the alkyl group contains 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, n-heptyl, and the like. In some embodiments, the alkyl group is methyl, ethyl, or propyl.

As used herein, “halo” or “halogen”, employed alone or in combination with other terms, includes fluoro, chloro, bromo, and iodo. In some embodiments, halo is F or Cl.

As used herein, the term “heterocycloalkyl,” employed alone or in combination with other terms, refers to a non-aromatic ring or ring system, which may optionally contain one or more alkenylene or alkynylene groups as part of the ring structure, which has at least one heteroatom ring member independently selected from nitrogen, sulfur, oxygen, and phosphorus. Heterocycloalkyl groups can include mono- or polycyclic (e.g., having 2, 3 or 4 fused, bridged, or spiro rings) ring systems. In some embodiments, the heterocycloalkyl group is a monocyclic or bicyclic group having 1, 2, 3, or 4 heteroatoms independently selected from nitrogen, sulfur and oxygen. Also included in the definition of heterocycloalkyl are moieties that have one or more aromatic rings (e.g., aryl or heteroaryl rings) fused (i.e., having a bond in common with) to the non-aromatic heterocycloalkyl ring, for example, 1,2,3,4-tetrahydro-quinoline and the like. Heterocycloalkyl groups can also include bridgehead heterocycloalkyl groups (e.g., a heterocycloalkyl moiety containing at least one bridgehead atom, such as azaadmantan-1-yl and the like) and spiroheterocycloalkyl groups (e.g., a heterocycloalkyl moiety containing at least two rings fused at a single atom, such as [1,4-dioxa-8-aza-spiro[4.5]decan-N-yl] and the like). In some embodiments, the heterocycloalkyl group has 3 to 10 ring-forming atoms, 4 to 10 ring-forming atoms, or about 3 to 8 ring forming atoms. In some embodiments, the heterocycloalkyl group has 2 to 20 carbon atoms, 2 to 15 carbon atoms, 2 to 10 carbon atoms, or about 2 to 8 carbon atoms. In some embodiments, the heterocycloalkyl group has 1 to 5 heteroatoms, 1 to 4 heteroatoms, 1 to 3 heteroatoms, or 1 to 2 heteroatoms. The carbon atoms or heteroatoms in the ring(s) of the heterocycloalkyl group can be oxidized to form a carbonyl, an N-oxide, or a sulfonyl group (or other oxidized linkage) or a nitrogen atom can be quaternized. In some embodiments, the heterocycloalkyl portion is a C₂₋₇ monocyclic heterocycloalkyl group. In some embodiments, the heterocycloalkyl group is a morpholine ring, pyrrolidine ring, piperazine ring, piperidine ring, tetrahydropyran ring, tetrahyropyridine, azetidine ring, or tetrahydrofuran ring.

As used herein, the term “heteroaryl,” employed alone or in combination with other terms, refers to a monocyclic or polycyclic (e.g., a fused ring system) aromatic hydrocarbon moiety, having one or more heteroatom ring members independently selected from nitrogen, sulfur and oxygen. In some embodiments, the heteroaryl group is a monocyclic or a bicyclic group having 1, 2, 3, or 4 heteroatoms independently selected from nitrogen, sulfur and oxygen. Example heteroaryl groups include, but are not limited to, pyridyl, pyrimidinyl, pyrazinyl, pyridazinyl, triazinyl, furyl, thienyl, imidazolyl, thiazolyl, pyrryl, oxazolyl, isoxazolyl, pyrazolyl, triazolyl, tetrazolyl, 1,2,4-thiadiazolyl, isothiazolyl or the like. The carbon atoms or heteroatoms in the ring(s) of the heteroaryl group can be oxidized to form a carbonyl, an N-oxide, or a sulfonyl group (or other oxidized linkage) or a nitrogen atom can be quaternized, provided the aromatic nature of the ring is preserved. In some embodiments, the heteroaryl group has 1 to 4, 1 to 3, or 1 to 2 heteroatoms.

### Methods of Use

Compound 1 and salts thereof exhibit affinity for S1P receptors. In particular, compounds of the invention show selective affinity for the S1P5 receptor over the S1P1 and/or S1P3 receptor(s).

Compound 1 and salts thereof are modulators of the S1P receptor, in particular of the S1P5 receptor. More specifically, the compounds and salts of the invention are S1P5 receptor agonists. The compounds and salts of the invention are useful for treating, alleviating and preventing diseases associated with S1P receptors (e.g., S1P5) or in which modulation of the endogenous S1P signaling system via any S1P receptor is involved. In particular, the compounds and salts of the present invention may be used to treat, alleviate or prevent CNS (central nervous system) disorders, such as neurodegenerative disorders, in particular, but not limited to, cognitive disorders (in particular age-related cognitive decline) and related conditions such as, e.g., Alzheimer's disease, (vascular) dementia, Nieman's Pick disease, and cognitive deficits in schizophrenia, obsessive-compulsive behavior, major depression, autism, multiple sclerosis and pain. Preferably, the compounds and salts of the present invention may be used to treat, alleviate or prevent cognitive disorders (in particular age-related cognitive decline) and related conditions.

As used herein, the term “contacting” refers to the bringing together of the indicated moieties in an in vitro system or an in vivo system such that they are in sufficient physical proximity to interact.

The terms “individual” or “patient,” used interchangeably, refer to any animal, including mammals, such as humans, mice, rats, other rodents, rabbits, dogs, cats, swine, cattle, sheep, horses, and primates. In some embodiments, the individual or patient is a human.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

It is appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination.

### Combination Therapy

One or more additional pharmaceutical agents or treatment methods can be used in combination with Compound 1 or a salt thereof for treatment of S1P receptor-associated diseases, disorders, or conditions, or diseases or conditions as described herein. The agents can be combined with the present compounds in a single dosage form, or the agents can be administered simultaneously or sequentially as separate dosage forms. In some embodiments, the additional pharmaceutical agent is an anti-Alzheimer's drug. In some embodiments, the additional pharmaceutical agent is an anti-vascular dementia drug. In some embodiments, the additional pharmaceutical agent is a cholinesterase inhibitor (e.g., donepezil, galantamine, and rivastigmine), N-methyl-D-aspartate receptor antagonist, memantine, nimodipine, hydergine, nicergoline, CDP-choline, or folic acid.

In some embodiments, the additional pharmaceutical agent is an anti-psychotic. In some embodiments, the additional pharmaceutical agent is chlorpromazine, fluphenazine, haloperidol, perphenazine, aripiprazole, asenapine, brexpiprazole, cariprazine, clozapine, lloperidone, lurasidone, olanzapine, paliperidone, quetiapine, risperidone, or ziprasidone.

### Pharmaceutical Formulations and Dosage Forms

When employed as pharmaceuticals, the compounds and salts of the present disclosure can be administered in the form of pharmaceutical compositions. Thus the present disclosure provides a composition comprising a compound or salt as described herein, a compound or salt as recited in any of the claims and described herein, or any of the embodiments thereof, and at least one pharmaceutically acceptable carrier. These compositions can be prepared in a manner well known in the pharmaceutical arts, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Pharmaceutical preparations may include amorphous solid dispersions prepared by spray drying or by hot melt extrusion with pharmaceutical acceptable polymers.

This invention also includes pharmaceutical compositions which contain, as the active ingredient, the compound or salt of the present disclosure or a pharmaceutically acceptable salt thereof, in combination with one or more pharmaceutically acceptable carriers. In some embodiments, the composition is suitable for topical administration. In making the compositions of the invention, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In some embodiments, the composition is a sustained release composition comprising at least one compound or salt described herein, or a pharmaceutically acceptable salt thereof, and at least one pharmaceutically acceptable carrier or excipient.

In some embodiments, the composition is prepared as a solid solution or a solid dispersion. It is an amorphous system prepared by hot melt granulation or by hot melt extrusion. The amorphous solid dispersion may be prepared by spray drying with a variety of pharmaceutical polymers. The amorphous solid dispersion may be prepared into a tablet or other alternative dosage forms for oral or alternative routes of administration.

The compositions can be formulated in a unit dosage form, each dosage containing from about 5 to about 1,000 mg (1 g). The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound may be effective over a wide dosage range and is generally administered in a therapeutically effective amount. It will be understood, however, that the amount of the compound or salt actually administered will usually be determined by a physician, according to the relevant circumstances, including the condition to be treated, the chosen route of administration, the actual compound or salt administered, the age, weight, and response of the individual patient, the severity of the patient's symptoms and the like.

The therapeutic dosage of a compound or salt of the present invention can vary according to, e.g., the particular use for which the treatment is made, the manner of administration of the compound or salt, the health and condition of the patient, and the judgment of the prescribing physician. The proportion or concentration of a compound or salt of the invention in a pharmaceutical composition can vary depending upon a number of factors including dosage, chemical characteristics (e.g., hydrophobicity), and the route of administration. The dosage is likely to depend on such variables as the type and extent of progression of the disease or disorder, the overall health status of the particular patient, the relative biological efficacy of the compound or salt selected, formulation of the excipient, and its route of administration. Effective doses can be extrapolated from dose-response curves derived from in vitro or animal model test systems.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers.

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination.

### EXAMPLES

### Experimental Methods

All reactions were run in glass or glass-lined steel equipment. Products were isolated and dried in an agitated filter/dryer (Hastelloy). In addition, isolation could be performed in any fixed-plate filter (e.g., Nutsche or Aurora) or a centrifuge, and drying done in a tray dryer. NMR spectra were collected on a Bruker 400 MHz NMR.

### Example 1: Synthesis of Compound 1

### Step 1. tert-butyl 2-(4-((4-chlorobenzyl)oxy)phenyl)-6,7-dihydrooxazolo[4,5-c]pyridine-5(4H)-carboxylate (Compound 4)

[chemical structure]

Acetonitrile (15.6 kg) was charged to an inerted vessel, followed by tert-butyl 2-(4-hydroxyphenyl)-6,7-dihydrooxazolo[4,5-c]pyridine-5(4H)-carboxylate (1.0 kg, limiting reagent; see U.S. Pat. No. 8,796,262 at col. 44), p-chlorobenzyl bromide (740 g, 1.14 equiv.), and powdered potassium carbonate (880 g, 1.06 mol-equiv). The mixture was heated to 50±5° C. and agitated at that temperature until the starting material was consumed, as judged by HPLC. The mixture was cooled to 25±5° C., whereupon water (USP purified, 40 kg) was added. After agitating for 1 hour, the product was filtered and washed with water (USP purified, 4 kg).

The wet product was reslurried in water (USP purified, 15 kg) for at least 1 hour at ambient temperature, filtered, and washed with water (USP purified, 5 kg). The product was dried at 50±5° C. under ≥26 in-Hg until the KF is ≤1%, at least 24 hours. The yield of the product was about 1.18 kg (85%).

### Step 2. 2-(4-((4-chlorobenzyl)oxy)phenyl)-4,5,6,7-tetrahydrooxazolo[4,5-c]pyridine (Compound 2)

[chemical structure]

tert-butyl 2-(4-((4-Chlorobenzyl)oxy)phenyl)-6,7-dihydrooxazolo[4,5-c]pyridine-5(4H)-carboxylate (1.0 kg, limiting reagent) was charged to an inerted vessel, followed by methylene chloride (13.3 kg) and trifluoroacetic acid (2.64 kg, ca. 10 equiv.). The mixture was stirred at 25±5° C. until the starting material was consumed, as judged by HPLC, which was at least 16 hours. The reaction was concentrated under vacuum to the minimum stirrable volume (MSV), after which ethyl acetate (4 kg) is added. The distillation was continued to MSV, and again ethyl acetate (4 kg) was added. The reaction was concentrated to MSV one final time, after which ethyl acetate (7.2 kg) was again added. A suspension formed. After stirring for 15 minutes, sodium bicarbonate (1.03 kg) in water (USP purified, 10.45 kg) was added until the pH of the aqueous layer was 7.0-8.5. The suspension was stirred for 15 more minutes, after which the pH was confirmed to be 7.0-8.5. The product was filtered and washed with water (USP purified, 4.0 kg).

The wet product was reslurried in water (USP purified, 10 kg) for at least 1 hour at ambient temperature, filtered, and washed with water (USP purified, 10 kg). The product was dried at 50±5° C. under ≥26 in-Hg until the KF is 1%, at least 24 hours. The yield was about 0.696 kg (90%).

### Step 3. (1s,3s)-3-(2-(4-((4-chlorobenzyl)oxy)phenyl)-6,7-dihydrooxazolo[4,5-c]pyridin-5(4H)-yl)cyclobutane-1-carboxylic acid (Compound 1)

[chemical structure]

2-(4-((4-Chlorobenzyl)oxy)phenyl)-4,5,6,7-tetrahydrooxazolo[4,5-c]pyridine (1.0 kg, limiting reagent) was added to an inerted vessel, followed by methanol (12.7 kg) and 3-oxo-cyclobutane-1-carboxylic acid (0.413 kg, 1.24 equiv.). The resulting mixture was stirred at 25±5° C. for a minimum of 2 hours. Then a solution of sodium cyanoborohydride (0.399 kg, 2.17 mol-equiv.) in methanol (2.50 kg) was added at a rate that maintains the temperature below 35° C. The addition vessel was rinsed with methanol (0.74 kg) and the rinse added to the reaction. Stirring was continued without the addition of heat (20-35° C.) until an IPT showed that the sum of the concentrations of starting material and imine was consumed, as judged by HPLC. The product, which had precipitated, was filtered and washed with methanol (4.75 kg) and water (USP purified, 6.03 kg). The product was dried at 50±5° C. under ≥26 in-Hg until the KF is 1%, at least 24 hours. The yield of crude product was about 1.09 kg (85%).

### Step 4. Recrystallization from DMSO

The crude product (1.0 kg) of Step 3 was charged to an inerted vessel, followed by DMSO (26.8 kg). The mixture was heated to 70±5° C., at which point a solution was obtained. The mixture was cooled as close to 20° C. as possible. The crystallized product was filtered and washed with three portions of methanol (3.2 kg each). The product was dried at 50±5° C. under ≥26 in-Hg.

### Step 5. Recrystallization from Acetic Acid/Water

The product from Step 4 (1.0 kg) was charged to an inerted vessel, followed by acetic acid (6.29 kg). The mixture was heated to 70-75° C., at which point a solution was obtained. The solution was filtered through a 0.2-micron cartridge filter and the temperature of the filtrate readjusted to 70-75° C., if necessary. Water (USP purified, 0.85 kg) was added, followed by 2 wt % of ESB1609 seeds. The mixture was stirred at 70-75° C. for about 30 minutes. Then additional water (USP purified, 0.85 kg) as added over about 2 hours while maintaining the temperature at 70-75° C. Next, the batch was cooled at about 0.6° C./min to 20±3° C., where it was agitated for at least 12 hours. The product was filtered, washed with water (USP purified, 2.0 kg) and dried at 35±5° C. with a nitrogen purge until the level of residual acetic acid was <5000 ppm. The solid product was then re-equilibrated to form the monohydrate.

Alternatively, the crude product from Step 3 can be used directly in the recrystallization form acetic acid/water to provide Compound 1 as a solid.

An NMR spectrum of Compound 1 in deuterated DMSO is depicted in FIG. 1.

### Example 2: Alternative Preparation of tert-butyl 2-(4-hydroxyphenyl)-6,7-dihydrooxazolo[4,5-c]pyridine-5(4H)-carboxylate (Compound 5)

### Step 1: 4-(chlorocarbonyl)phenyl acetate (Compound 9)

[chemical structure]

p-Hydroxybenzoic acid was reacted with acetic anhydride and triethylamine at 100° C. for 6 h to provide 4-acetoxybenzoic acid. After oven drying, 4-acetoxybenzoic acid was mixed with oxalyl chloride in methylene chloride at room temperature for about 6 hours to give 4-(chlorocarbonyl)phenyl acetate in quantitative yield.

### Step 2: tert-butyl 4-((tosyloxy)imino)piperidine-1-carboxylate (Compound 13)

[chemical structure]

N-Boc-piperidin-4-one was mixed with hydroxylamine HCl and sodium hydroxide in water/MeOH at room temperature for 8 hours. The resulting product, tert-butyl 4-(hydroxyimino)piperidine-1-carboxylate, was collected by filtration as a white solid and, after drying, was isolated in 95% yield. Subsequently, tert-butyl 4-(hydroxyimino)piperidine-1-carboxylate was dissolved in methylene chloride and treated with triethylamine and tosyl chloride to give tert-butyl 4-((tosyloxy)imino)piperidine-1-carboxylate. After removal of the solvent under vacuum, tert-butyl 4-((tosyloxy)imino)piperidine-1-carboxylate was obtained as a white solid.

### Step 3: tert-butyl 3-amino-4,4-diethoxypiperidine-1-carboxylate

[chemical structure]

tert-Butyl 4-((tosyloxy)imino)piperidine-1-carboxylate was suspended in ethanol and a solution of sodium ethoxide in ethanol was added, keeping the temperature below 10° C. After 8 h, the ethanol was removed under reduced pressure, water was added, and the product was extracted with ethyl acetate, with an estimated yield of tert-butyl 3-amino-4,4-diethoxypiperidine-1-carboxylate of 65%. The ethyl acetate solution of tert-butyl 3-amino-4,4-diethoxypiperidine-1-carboxylate was used directly in the next step.

### Step 4: tert-butyl 3-(4-acetoxybenzamido)-4-oxopiperidine-1-carboxylate (Compound 7)

[chemical structure]

The ethyl acetate solution of tert-butyl 3-amino-4,4-diethoxypiperidine-1-carboxylate was mixed with triethylamine, followed by the addition of compound 4-(chlorocarbonyl)phenyl acetate. When the reaction was complete (as judged by HPLC), it was washed with water to remove triethylamine hydrochloride. Then, the organic solution containing tert-butyl 3-(4-acetoxybenzamido)-4,4-diethoxypiperidine-1-carboxylate was mixed with 3N HCl to give tert-butyl 3-(4-acetoxybenzamido)-4-oxopiperidine-1-carboxylate. After removal of ethyl acetate by vacuum, the crude tert-butyl 3-(4-acetoxybenzamido)-4-oxopiperidine-1-carboxylate was obtained as gum and used without any purification for the next step.

Alternatively, tert-butyl 4-(hydroxyimino)piperidine-1-carboxylate was dissolved in methylene chloride and treated with triethylamine and tosyl chloride, keeping the temperature below 10° C. The byproduct triethylamine hydrochloride was removed by filtration and the resulting methylene chloride solution of tert-butyl 4-((tosyloxy)imino)piperidine-1-carboxylate was treated with potassium methoxide in methanol at approximately 5° C. for 6 h, followed by triethylamine and 4-(chlorocarbonyl)phenyl acetate below 10° C. The reaction was monitored by HPLC. The reaction was washed with water and the organic phase containing tert-butyl 3-amino-4,4-diethoxypiperidine-1-carboxylate was treated with 3N HCl to provide tert-butyl 3-(4-acetoxybenzamido)-4-oxopiperidine-1-carboxylate.

### Step 5: tert-butyl 2-(4-hydroxyphenyl)-6,7-dihydrooxazolo[4,5-c]pyridine-5(4H)-carboxylate (Compound 5)

[chemical structure]

tert-Butyl 3-(4-acetoxybenzamido)-4-oxopiperidine-1-carboxylate was dissolved in acetonitrile. Pyridine, triphenylphosphine, and hexachloroethane were then added. When the reaction was complete, as measured by HPLC, the reaction mixture was diluted with water and filtered to collect the solids, which contained both the desired tert-butyl 2-(4-acetoxyphenyl)-6,7-dihydrooxazolo[4,5-c]pyridine-5(4H)-carboxylate and the byproduct triphenylphosphine oxide. The solid was mixed with 10% sodium hydroxide solution to hydrolyze the acetate group, and then, after filtering to remove triphenylphosphine oxide, the filtrate, containing the product as the sodium salt, was treated with 1N HCl, providing crude tert-butyl 2-(4-hydroxyphenyl)-6,7-dihydrooxazolo[4,5-c]pyridine-5(4H)-carboxylate (Compound 5). Recrystallization from ethanol gave the purified product in about 25% overall yield from Step 2. An NMR spectrum of Compound 5 in deuterated chloroform is depicted in FIG. 2.

Alternatively, to reduce the amount of triphenylphosphine oxide waste, phosphorous tribromide can be used in place of triphenylphosphine, as follows: tert-Butyl 3-(4-acetoxybenzamido)-4-oxopiperidine-1-carboxylate was dissolved in THF, followed by the addition of pyridine and phosphorus tribromide. When the reaction was judged complete by HPLC, the reaction was diluted with water. The organic phase containing tert-butyl 2-(4-acetoxyphenyl)-6,7-dihydrooxazolo[4,5-c]pyridine-5(4H)-carboxylate was mixed with 10% sodium hydroxide solution to hydrolyze the acetate. After separation of the organic phase, the aqueous phase (containing the product as the sodium salt) was treated with 1N HCl, and crude tert-butyl 2-(4-hydroxyphenyl)-6,7-dihydrooxazolo[4,5-c]pyridine-5(4H)-carboxylate was collected by filtration. The crude product was purified by recrystallization from ethanol, which provided tert-butyl 2-(4-hydroxyphenyl)-6,7-dihydrooxazolo[4,5-c]pyridine-5(4H)-carboxylate as an off-white solid. This new method significantly reduced the volume of waste, and provided Compound 1 in about 25% overall yield from Step 2.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A process for preparing Compound 1 having the formula: [chemical structure]
or a salt thereof, comprising reacting Compound 2, having the formula: [chemical structure]
with Compound 3, having the formula: [chemical structure]
in the presence of RA1, wherein RA1 is a reducing agent, to provide Compound 1, or a salt thereof.

2. The process of claim 1, wherein RA1 is a hydride reducing agent.

3. The process of claim 1, wherein RA1 is sodium cyanoborohydride.

4. The process of claim 1, wherein the reacting of Compound 2 with Compound 3 is performed in the presence of S1, wherein S1 is a protic solvent.

5. The process of claim 4, wherein S1 is methanol.

6. The process of claim 1, wherein the reacting of Compound 2 with Compound 3 comprises using about 1 to about 2 molar equivalents of Compound 3 relative to Compound 2.

7. The process of claim 1, wherein the reacting of Compound 2 with Compound 3 comprises using about 1 to about 3 molar equivalents of RA1 relative to Compound 2.

8. The process of claim 1, wherein the reacting of Compound 2 with Compound 3 is performed at room temperature.

9. The process of claim 1, further comprising precipitating Compound 1 from a solution comprising Compound 1 and Sla, wherein Sla is an aprotic solvent.

10. The process of claim 9, wherein Sla is DMSO.

11. The process of claim 1, further comprising precipitating Compound 1 from a solution comprising Compound 1 and S1b, wherein S1b is a mixture of a protic solvent and A1, wherein A1 is an acid.

12. The process of claim 11, wherein S1b is a mixture of water and A1.

13. The process of claim 11, wherein A1 is acetic acid.

14. The process of claim 1, wherein Compound 2 is prepared by a process comprising reacting Compound 4, having the formula: [chemical structure]
with A2, wherein A2 is an acid.

15. The process of claim 14, wherein A2 is an organic acid.

16. The process of claim 14, wherein A2 is trifluoroacetic acid.

17. The process of claim 14, wherein the reacting of Compound 4 with A2 is performed in the presence of S2, wherein S2 is a halogenated solvent.

18. The process of claim 17, wherein S2 is methylene chloride.

19. The process of claim 14, wherein the reacting of Compound 4 with A2 comprises using about 1 to about 20 molar equivalents of A2 relative to Compound 4.

20. The process of claim 14, wherein the reacting of Compound 4 with Compound A2 is performed at room temperature.

21. The process of claim 14, wherein Compound 4 is prepared by a process comprising reacting Compound 5, having the formula: [chemical structure]
with a compound of Formula II: [chemical structure]
wherein X is Br, Cl, or I, in the presence of B1, wherein B1 is a base.

22. The process of claim 21, wherein X is Br or Cl.

23. The process of claim 21, wherein X is Br.

24. The process of claim 21, wherein B1 is a carbonate base.

25. The process of claim 21, wherein B1 is potassium carbonate.

26. The process of claim 21, wherein the reacting of Compound 5 with the compound of Formula II in the presence of B1 is performed in the presence of S3, wherein S3 is a polar aprotic solvent.

27. The process of claim 26, wherein S3 is acetonitrile.

28. The process claim 21, wherein the reacting of Compound 5 with the compound of Formula II in the presence of B1 comprises using about 1 to about 5 molar equivalents of the compound of Formula II relative to Compound 5.

29. The process of claim 21, wherein the reacting of Compound 5 with the compound of Formula II in the presence of B1 comprises using about 1 to about 5 molar equivalents of B1 relative to Compound 5.

30. The process of claim 21, wherein the reacting of Compound 5 with the compound of Formula II in the presence of B1 is performed at a temperature between about 45° C. and about 55° C.

31. The process of claim 21, wherein Compound 5 is prepared by a process comprising reacting Compound 6 having the formula: [chemical structure]
with B4, wherein B4 is a base.

32. The process of claim 31, wherein B4 is sodium hydroxide.

33. The process of claim 31, wherein the reacting of Compound 6 with B4 can be performed in the presence of S4, wherein S4 is a polar solvent.

34. The process of claim 33, wherein S4 is water.

35. The process of claim 31, wherein Compound 6 is prepared by a process comprising reacting Compound 7 having the formula: [chemical structure]
with P1, wherein P1 is a phosphorous reagent.

36. The process of claim 35, wherein P1 is triphenylphosphine or phosphorous tribromide.

37. The process of claim 35, wherein the reacting of Compound 7 with P1 can be performed in the presence of S5, wherein S5 is a polar aprotic solvent.

38. The process of claim 37, wherein S5 is acetonitrile or THF.

39. The process of claim 35, wherein the reacting of Compound 7 with P1 is further performed in the presence of a mixture of pyridine and hexachloroethane.

40. The process of claim 35, wherein Compound 7 is prepared by a process comprising reacting Compound 8 having the formula: [chemical structure]
wherein R is C₁₋₂ alkyl;
with A3, wherein A3 is an acid.

41. The process of claim 40, wherein A3 is HCl.

42. The process of claim 40, wherein the reacting of Compound 8 with A3 can be performed in the presence of S6, wherein S6 is a polar aprotic solvent.

43. The process of claim 42, wherein S6 is ethyl acetate.

44. The process of claim 40, wherein R is ethyl.

45. The process of claim 40, wherein R is methyl.

46. The process of claim 40, wherein Compound 8 is prepared by a process comprising reacting Compound 9 having the formula: [chemical structure]
with Compound 12 having the formula: [chemical structure]
wherein R is C₁₋₂ alkyl, in the presence of B3, wherein B3 is a base.

47. The process of claim 46, wherein B3 is triethylamine.

48. The process of claim 46, wherein the reacting of Compound 9 with Compound 12 can be performed in the presence of S7, wherein S7 is a polar aprotic solvent.

49. The process of claim 48, wherein S7 is ethyl acetate.

50. The process of claim 46, wherein Compound 12 is prepared by a process comprising reacting Compound 13 having the formula: [chemical structure]
with sodium C₁₋₂ alkoxide.

51. The process of claim 50, wherein the sodium C₁₋₂ alkoxide is sodium methoxide.

52. The process of claim 50, wherein the sodium C₁₋₂ alkoxide is sodium ethoxide.

53. The process of claim 51, wherein the reacting of Compound 13 with sodium methoxide is performed in the presence of S10, wherein S10 is methanol.

54. The process of claim 52, wherein the reacting of Compound 13 with sodium ethoxide is performed in the presence of S10, wherein S10 is ethanol.

55. Compound 1, or a salt thereof, prepared by the process of claim 1.

56. A compound of Formula Ia: [chemical structure]
or a salt thereof, wherein R¹ is H, C₁₋₆ alkyl, C₃₋₇ cycloalkyl, phenyl, 4-7 membered heterocycloalkyl, or 5-6 membered heteroaryl, wherein the C₁₋₆ alkyl is optionally substituted by 1-5 halo, or optionally substituted by a C₃₋₇ cycloalkyl, phenyl, 4-7 membered heterocycloalkyl, or 5-6 membered heteroaryl group.

57. The compound of claim 56 wherein R¹ is C₁₋₆ alkyl.

58. A compound, having the following formula: [chemical structure]
or a salt thereof.

59. A compound, having the following formula: [chemical structure]
or a salt thereof.

60. A compound of Formula Ib: [chemical structure]
or a salt thereof, wherein R¹ is H, C₁₋₆ alkyl, C₃₋₇ cycloalkyl, phenyl, 4-7 membered heterocycloalkyl, or 5-6 membered heteroaryl, wherein the C₁₋₆ alkyl is optionally substituted by 1-5 halo, or optionally substituted by a C₃₋₇ cycloalkyl, phenyl, 4-7 membered heterocycloalkyl, or 5-6 membered heteroaryl group.

61. The compound of claim 60 wherein R¹ is C₁₋₆ alkyl.

62. A compound, having the following formula: [chemical structure]
or a salt thereof.
